(19) United States
(12) Patent Application Publication
(10) Pub. No.: US 2013/0197456 A1
(43) Pub. Date: Aug. 1, 2013

(54) Oil Resistant Polymer Device, Method and Kit for Treating Fungal Infected Nail

(71) Applicant: Yanhui Sun, Wilmington, DE (US)

(72) Inventor: Yanhui Sun, Wilmington, DE (US)

(21) Appl. No.: 13/362,904

(22) Filed: Jan. 31, 2012

(51) Int. Cl. A61M 35/00

(52) U.S. Cl. 604/290; 604/289; 604/293

(57) ABSTRACT

A device for treating at least one infected nail of at least one digit of a subject includes an enclosure sized for housing at least a distal end of at least one digit of a subject having at least one infected nail. The enclosure comprises an integrally molded elastomeric polymer sheath having an interior cavity, which substantially conforms to the size and shape of at least the distal end of the at least one digit of the subject having the at least one infected nail. The sheath has an open end and a closed end, tapering from the closed end to the open end. The elastomeric polymer sheath comprises an elastomeric polymer film having oil resistance and low oxygen permeability. A method of using the device to treat the infected nail is provided. A kit including the device and an antifungal treatment composition is disclosed for treating the infected nail.

### TECHNICAL FIELD

The presently disclosed and claimed inventive concept(s) relates generally to an oil resistant polymer device, a method and a kit for treating fungal infected nail(s).

### BACKGROUND

Fungi are among the strongest and most durable group of organisms. They are capable of growing in almost any place including a mammal. One class of fungal infections of mammals is dermatophytic infections, which are fungal infections of hair, nails, and skin.

Generally, fungal infections exhibit small, itchy enlarging papules that may resemble dermatophytosis which later extends to form dull red or violaceous, sharply demarcated patches with indurated bases. Hard, dull red or grayish cauliflower-shaped nodular projections may develop in the center of the patches, gradually extending to cover the extremities.

Dermatophytes are filamentous fungi that are commonly found in soil and plants. Once these organisms invade nails, they live on the nail bed and feed on keratin, which is fibrous structural protein. Water and free oxygen are also essential elements for these organisms to thrive.

Onychomycosis, also known as dermatophytic onychomycosis, ringworm of the nail, or tinea unguium, refers to a fungal infection of finger nails and toe nails. It is the most common nail disease and accounts for almost half of all nail dysfunctions. Toe nail infection is far more common than finger nail infection, probably due to the environment. The occurrence of toe nail infection is about 6-8% of the adult population. In the age group of 40 to 60 years, it grows to about 20%. The reported numbers have been increasing, possibly owing to factors such as diabetes, immune suppression, and increased life expectancy. Some patients start with athlete's foot infection. The infection spreads over to the toe nails as patients age if the infection is not well treated and controlled.

Onychomycosis exhibits visible skin or nail discoloration, lesions, neoplasms, or a combination thereof. In some cases of fungal infections, there may be a papule, a fixed subcutaneous nodule, a vesicle with an indurated base, or a subcutaneous abscess that ruptures to form a fistula to the skin surface.

Although it is not life threatening, onychomycosis can be painful and can cause serious damage to nails. Some older patients have difficulty walking due to the severity of the disease. It can also lead to other infections that could spread beyond patients' feet or hands if patients have suppressed immune systems due to medication, diabetes or other conditions.

Determatophyte infections of the skin and nails, caused by several species of fungi, have demonstrated a remarkable resilience against a growing array of antifungal agents developed to eliminate them. This is the consequence of factors intrinsic to the nail—the hard, protective nail plate, sequestration of pathogens between the nail bed and plate, and slow growth of the nail—as well as of the relatively poor efficacy of early pharmacologic agents.

Standard treatment for onychomycosis includes medications in different forms and surgical procedures. Oral medicines, such as terbinafine (LAMISIL®), itraconazole (SPORANOX®), and fluconazole (DIFLUCAN®) are commercially available for fungal nail treatment. Antifungal topical medicines, such as creams, lotions, gels, and lacquers, are applied to the infected nails and surrounding skin areas. Examples of topical medicines include terbinafine such as LAMISIL® and ciclopirox (PENLAC®). Removal of an infected nail is sometimes chosen for treating severe fungal infection and recurring infection. Nonsurgical nail removal involves application of urea ointment to soften and dissolve nail for easy removal.

Much research effort has been focused on improving the efficacy of topical medicines. Fibrous keratin protein, the structural component in nails, has lipid bilayers, phospholipid cell membrane and connections between the cells called desmosomes. The penetration of antifungal medicine has to rely on diffusion of non-polar lipid across this structure. To effectively treat onychomycosis, it is necessary for the medicine to penetrate through the nail plate and keratin to the site where organisms live. This has proven to be difficult and tricky.

Even with the advancement in the pharmaceutical field, drug delivery area, and assistant technologies, onychomycosis treatment remains quite challenging, especially for patients with existing conditions. Clearly, there is still a need to provide an effective, safe, easily applied, and inexpensive device for treating onychomycosis and other function infections.

### BRIEF SUMMARY OF THE DRAWINGS

FIG. 1 illustrates a perspective view of an oil resistant polymer device constructed and operated onto an infected toe nail in accordance with one embodiment of the presently disclosed and claimed inventive concept(s).

FIG. 2 illustrates a perspective view of an oil resistant polymer device constructed and operated onto an infected finger nail in accordance with one embodiment of the presently disclosed and claimed inventive concept(s).

FIG. 3 illustrates a perspective view of an oil resistant polymer device constructed and operated onto an infected forefoot in accordance with one embodiment of the presently disclosed and claimed inventive concept(s).

### DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of chemistry described herein are those well known and commonly used in the art. Reactions and purification techniques are performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analysis, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, and/or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

In addition, as used herein and throughout, the term “digit” generally refers to any one of multiple appendages or toes on a mammal's foot, including the hallux, or any of multiple digits on a mammal's hand, including the thumb.

As used herein and throughout, the term “toe” means and refers to any one of multiple appendages or digits on a mammal's or a person's foot including the hallux.

As used herein and throughout, the term “finger” means and refers to any one of multiple digits on a mammal's or a person's hand including the thumb.

As used herein, the terms “elastic” and “elastomeric” are generally used to refer to materials that, upon application of a force, are stretchable to a stretched, biased length which is at least about 125%, or one and one fourth times, its relaxed, unstretched length, and which will retract at least about 50% of its elongation upon release of the stretching, biasing force.

The term “treating” or “treatment” as used herein encompasses any treatment of onychomycosis or fungal infections, and includes:

preventing the disease from occurring in a subject which may be predisposed to the disease but has not yet been diagnosed as having it;

inhibiting the disease, i.e., arresting its development; and/or relieving the disease, i.e., causing regression of the disease.

In the context of the presently disclosed and claimed inventive concept(s), relieving the disease means attaining improvement in the subject's condition, including, but not limited to clinical improvement, microbiological improvement, and aesthetic improvement.

The presently disclosed and claimed inventive concept(s) relates to a deceptively simple, yet effective and novel, device for treating fungal infected nails. In one embodiment, the device can be used for treating at least one infected nail of at least one digit of a subject. The subject can be a mammal, particularly a human. The device is resistant to oil and low permeable to gas, more particularly oxygen. The oil resistant polymer device comprises an enclosure. The enclosure is designed and sized least one digit of a subject having at least one infected nail. In one embodiment, the enclosure comprises an integrally molded elastomeric polymer sheath. The sheath has an interior cavity, which substantially conforms to the size and shape of the at least distal end of the at least one digit of the subject. The sheath has an open end and a closed end.

The elastomeric polymer sheath is constructed of an elastomeric polymer film having oil resistance and low oxygen permeability. The elastomeric polymer film can provide the sheath with form-fitting properties. In one embodiment, the sheath is tapered from the closed end to the open end. The open end of the sheath allows at least the distal end(s) of the digit(s) of the subject that contain an infected nail(s) to be inserted into the interior cavity so that the closed end can cover the infected nail(s). As a result, the enclosure is tightly sealed around the distal end(s) of the digits(s) containing the infected nail(s), but the internal circulation to the nail(s) is not affected.

The elastomeric polymer film utilized in accordance with the presently disclosed and claimed inventive concept(s) may be any elastomeric polymer resistant to oil and low permeable to oxygen and capable of use as described herein. Examples of such elastomeric polymers can include, but are not limited to, polybutadiene-co-acrylonitrile, polystyrene terpolymer with butadiene and acrylonitrile, chloroprene rubber, halogenated butyl rubber, epichlorohydrin rubber, polyacrylic esters, fluoroelastomers, perfluoroelastomers, chlorosulfonated polyethylene, polyether-amide, polyether-ester, polyurethane, polyisoprene-co-acrylonitrile, and combinations thereof.

In one embodiment, an elastomeric polymer film with oil resistance and low oxygen permeability comprises polybutadiene-co-acrylonitrile. Polybutadiene-co-acrylonitrile, also known as a nitrile polymer, has been widely used in automotive and aeronautical industries. It can provide excellent resistance to oils and acids, and has a good resistance to puncturing.

Generally, polymers with polar groups (i.e. polar polymers), such as polyvinyl alcohol, polyamide homopolymer, polyvinylidene chloride, polyvinylidene, polyethylene terephthalate, and polyacrylonitrile possess excellent oil resistances. However, these are thermoplastic polymers, which normally have glass transition temperatures above room temperature. At temperatures below their glass transition temperatures, the polymers are brittle and less pliable. Structural modifications of these polymers are often necessary to obtain the desired elastomeric properties. The modifications include, but are not limited to, copolymerization and polymer blending. In one embodiment, an elastomeric polymer film with oil resistance and low oxygen permeability comprises copolymers and/or blends of polyvinyl alcohol, polyamide, polyvinylidene chloride, polyvinylidene, polyacrylonitrile, and polyethylene terephthalate_containing soft segments and/or soft chains to reduce their glass transition temperatures to much lower than room temperature. For example, polyacrylonitrile is a hard, rigid thermoplastic resin. However, when polyacrylonitrile is copolymerized with butadiene, it becomes very pliable and often serves as an elastomer for many applications.

As another example, polyamide has a melting point at 265° C. and is a perfect engineering polymer for automotive parts due to its highly rigid and polar polymer chain. However, polyamide has to be modified if elasticity and softness are required in the application. A common way to increase its flexibility is to copolymerize polyamide with polyether segments to form polyetheramide block copolymers. The copolymers have excellent elasticity and function as good thermoplastic elastomers. Polyethylene terephthalate can also be modified by incorporating polyether segments into its chain to form block copolymers.

Low oxygen permeability, as used herein, generally refers to an oxygen permeation coefficient of film of less than about 5×10⁻¹³cm³×cm/cm²×s×Pa measured at 25° C. according to ASTM D1434-82, procedure V-Volumetric (Standard Method for Determining Gas Permeability Characteristics of Plastic Film and Sheeting, 1999) and Journal of Applied Physics, Vol. 17, 972-985 (1946), the entirety of which are hereby expressly incorporated herein by reference. Permeation coefficient is defined as the volume of gas which, under steady conditions, crosses unit area and unit thickness of a film in unit time under unit pressure difference and at constant temperature as shown below,

P=(quantity of permeation)(film thickness)/(area)(time) (pressure drop across the film).

In one embodiment, the elastomeric polymer film has an oxygen permeation coefficient of less than about 3×10⁻¹³cm³×cm/cm²×s×Pa. In another embodiment, the film has an oxygen permeation coefficient between about 0.05×10⁻¹³ and about 2.5×10⁻¹³ cm³×cm/cm²×s×Pa.

An elastomeric polymer sheath with an interior cavity in the presently disclosed and claimed inventive concept(s) is generally fabricated from an emulsion-based elastomeric polymer (e.g., polymers formed by emulsion polymerization). The sheath can be formed by any method known in the art or otherwise described herein; for example but not by way of limitation, the sheath may be formed on a forming apparatus (i.e., a former). In one embodiment, the sheath can be formed through a series of dipping or immersions of the former. For example, in one particular embodiment, a sheath-shaped former, such as but not limited to a ceramic former, is dipped into a coagulant bath that facilitates later stripping of a sheath from the former. The coagulant bath may contain a coagulant composition comprising calcium carbonate and/or calcium nitrate. Thereafter, the coagulant-coated former is dried and subsequently dipped into one or more emulsion baths. The former can be removed from the emulsion baths. The resulting elastomeric polymer layer(s) are then typically leached in water to extract a large percentage of the water-soluble impurities in the elastomeric polymer and coagulant. The coated former is dried to cure (i.e., crosslink) the elastomeric polymer. The sheath can then be stripped from the former.

The sheath made in the presently disclosed and claimed inventive concept(s) comprises a thin film of elastomeric polymer. The elastomeric polymeric film may be provided with any thickness that allows the elastomeric polymeric film to function in accordance with the presently disclosed and claimed inventive concept(s). For example but not by way of limitation, the elastomeric polymer film may be provided with a thickness that is less than about 0.3 mm. In one embodiment, the film can be between about 0.1 and about 0.2 mm thick. It should be understood that the conditions, process, and materials used in forming elastomeric polymer sheath are well known in the art, and are not critical to the practice of the presently disclosed and claimed inventive concept(s). Therefore, no further discussion of the formation of the elastomeric polymer sheath is deemed necessary.

An oil resistant elastomeric polymer sheath is fabricated to fit and tightly seal at least a portion (i.e., a distal end) of at least one digit of a subject having an infected nail, and may also fit and tightly seal at least a portion of the surrounding skin areas as well. The dimensions of the sheath formed in accordance with the presently disclosed and claimed inventive concept(s) will depend upon a particular application for which the sheath is to be used. For instance, the sheath can be constructed in order to fit around one finger or one toe. Further, the sheath can be constructed to fit around two or more fingers or toes. The sheath can also be made to fit a fungal infected portion of a forefoot. As used herein, the term “forefoot” is intended to denote that portion of a foot which is maximally responsible for propulsive contact of the foot with the support surface and may be broadly anatomically defined as that portion of the foot existing between the distal ends of the metatarsals and the distal ends of the phalanges. In one embodiment, the sheathe can be made to fit a fungal infection portion collectively covering four fingers of a hand, namely the index finger, middle finger, ring finger and little finger of the hand.

In one specific embodiment, the interior cavity can contain an antifungal treatment composition and a wax. The antifungal treatment composition is mixed with the wax. In another specific embodiment, the interior cavity can contain an antifungal treatment composition absorbed in a polymer gel.

In one embodiment, the antifungal treatment composition is an antifungal medicine for topical application. Topical antifungal medicine include, but are not limited to, miconazole, ketoconazole, econazole, oxiconazole, clotrimazole, naftifine, terbinafine and undecylenic acid, imidazole derivatives, amorolfine, saperconazole, pharmaceutically acceptable salts and stereoisomers, bifonazole plus urea, other antifungal drugs that are available in a topical formulation, and combinations thereof.

The antifungal treatment composition is usually dissolved in an oleophilic solvent to form a solution. The solution is then mixed with the wax to form a mixture. The antifungal treatment composition of the device ranges from about 1% to about 35% by weight based on the total amount of the mixture. In one embodiment, the antifungal treatment composition ranges from about 1% to about 25% by weight based on the total amount of the mixture. The mixture is added into the interior cavity of the elastomeric polymer sheath. The wax ensures that a thin layer of the antifungal medicine can be evenly distributed over an infected nail.

As used herein, the term “wax” includes, but is not limited to, any hydrophobic material that is: solid at 25° C.; practically insoluble in water according to the United States' Pharmacopeia (USP) definition in 31/NF 26 Vol. 2 General Notices, Page Xvii.; has an onset temperature measured according to the DSC Method, defined herein below, which is 10° C. or greater; and comprises lipids, silicones or mixtures thereof. The wax may include natural wax, synthetic wax, silicone wax, or mixtures thereof.

Examples of suitable natural waxes include, but are not limited to, Abies Alba Leaf Wax, Acacia Dealbata Leaf Wax, Acacia Farnesiana Flower Wax, Beeswax, Ceresin, Cetyl Esters, Cistus Labdaniferus Flower Wax, Aurantium Amara (Bitter Orange) Flower Wax, Aurantium Dulcis (Orange) Peel Wax, Copernicia Cerifera (Carnauba) Wax, Eclipta Prostrata Wax, Euphorbia Cerifera (Candelilla) Wax, Helichrysum Angustifolium Wax, Jasminum Officinale (Jasmine) Flower Wax, Jasminum Sambac (Jasmine) Flower Wax, Jojoba Esters, Jojoba Wax, Lanolin Wax, Lavandula Angustifolia (Lavender) Flower Wax, Lawsonia lnermis Wax, Mink Wax, Montan Acid Wax, Montan Wax, Myrica Cerifera (Bayberry) Fruit Wax, Ocimum Tenuiflorum Wax, Olive Wax, Oryza Sativa (Rice) Bran Wax, Ouricury Wax, Palm Kernel Wax, Persea Gratissima (Avocado) Wax, Pistacia Lentiscus Leaf Wax, Polianthes Tuberosa Flower Wax, Pyrus Malus (Apple) Peel Wax, Ribes Nigrum (Black Currant) Wax, Rosa Centifolia Flower Wax, Salvia Sclarea (Clary) Wax, Shellac Wax, Simmondsia Chinensis (Jojoba) Butter, Soft Olive Wax, Spent Grain Wax, Stipa Tenacissima Wax, Sunflower Seed Wax, Vegetable Wax, Vitis Vinifera (Grape) Leaf Wax and mixtures thereof.

Examples of suitable synthetic waxes include, but are not limited to, Hydrogenated Japan Wax, Hydrogenated Jojoba Oil, Hydrogenated Jojoba Wax, Hydrogenated Microcrystalline Wax, Hydrogenated Rice Bran Wax, Hydrolyzed Beeswax, Microcrystalline Wax, Oxidized Beeswax, Oxidized Microcrystalline Wax, Ozokerite, Paraffin, PEG-6 Beeswax, PEG-8 Beeswax, PEG-12 Beeswax, PEG-20 Beeswax, PEG-12 Carnauba, Potassium Oxidized Microcrystalline Wax, Sulfurized Jojoba Oil, Synthetic Beeswax, Synthetic Candelilla Wax, Synthetic Carnauba, Synthetic Japan Wax, Synthetic Jojoba Oil, Synthetic Wax and mixtures thereof.

Examples of suitable silicone waxes include, but are not limited to, DC2503 Cosmetic Wax, DC580 wax, DC AMS-C30 Cosmetic Wax, C30-45 Alkyl Methicone, DC Silkywax 10, Hexamethyldisiloxane, DC ST-Wax 30, C30-45 Alkyldimethylsilyl Polypropylsilsesquioxane, DC SW-8005 resin wax, C26-28 Alkyl Dimethicone, C26-28 Alkyl Methicone, Polyphenylsilsesquioxane and mixtures thereof.

The presently disclosed and claimed inventive concept(s) provides a method of treating at least one infected nail of at least one digit of a subject using the device described previously. The method comprises: (a) topically administering an effective amount of an antifungal treatment composition to at least a distal end of at least one digit of a subject having at least on infected nail; (b) disposing a device over the at least distal end of the at least one digit of the subject having the at least one infected nail, the device comprising an enclosure sized for housing the at least distal end of the at least one digit of the subject having the at least one infected nail, the enclosure comprising an integrally molded elastomeric polymer sheath having an interior cavity, the interior cavity substantially conforming to the size and shape of the at least distal end of the at least one digit of the subject having the at least one infected nail, and the sheath having an open end and a closed end and tapering from the closed end to the open end; and (c) tightly sealing the device around the at least distal end of the at least one digit of the subject having the at least one infected nail.

The methods of the presently disclosed and claimed inventive concept(s) can be used to reduce, treat, or remove the exterior blemishes caused by fungal infections. In particular, the method can be used to improve the exterior skin appearance and remove physical signs associated with fungal infections. As used herein, the term “improve skin appearance” refers to removal or diminishment in appearance or size of skin or nail discolorations and skin blemishes associated with fungal infections.

In one embodiment, the antifungal treatment composition is an antifungal medicine for topical application. The antifungal medicine utilized in accordance with the methods of the presently disclosed and claimed inventive concept(s) is in the form of liquid. The antifungal medicine can usually be dissolved in an oleophilic solvent in order to obtain better penetration through the lipid bilayer in keratin, which is the main component of nails. The topical antifungal medicine used in the method is the same as those described above herein.

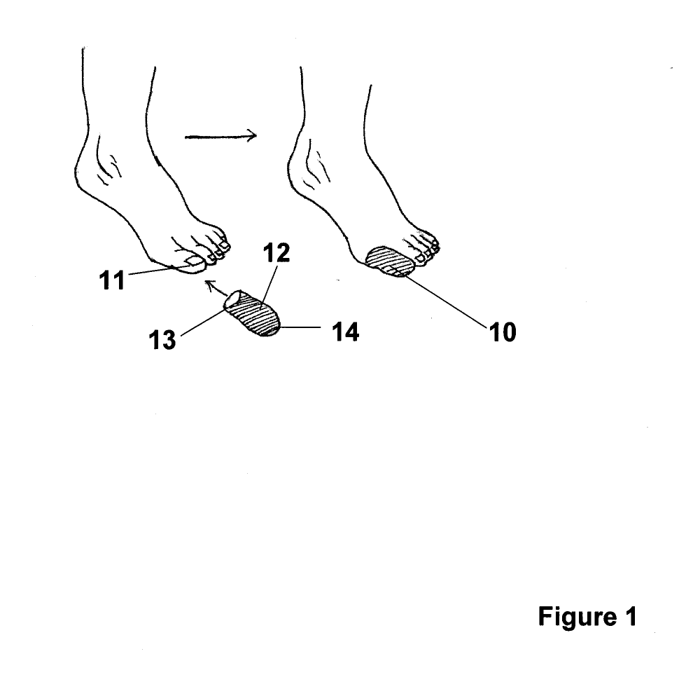

Referring to FIGS. 1, 2 and 3, various exemplary embodiments of an oil resistant elastomeric polymer device constructed and operated in accordance with the presently disclosed and claimed inventive concept(s) are disclosed. For illustrative purposes, an oil resistant elastomeric polymer device of the presently disclosed and claimed inventive concept(s) is described herein as a device to fit various infection areas of a subject. However, it should be understood that an oil resistant elastomeric polymer device of the presently disclosed and claimed inventive concept(s) is not limited to be used for these infected areas, and it can also be made to fit other infections inserted therein.

FIG. 1 illustrates an oil resistance polymer device 10 before and after its application to an infected toe nail 11. In the exemplary embodiment, the device 10 comprises an enclosure sized for housing the toe nail 11. The enclosure includes an integrally molded elastomeric polymer sheath having an interior cavity 12, an open end 13 and a closed end 14. The interior cavity 12 substantially conforms to the size and shape of the toe nail 11 and its surrounding skin areas. The device 10 may have a slightly tapered shape such that the open end 13 is narrower than the closed end 14. Such a tapered shape allows the device 10 to better fit the toe nail 11 with a substantially tight seal, but not too tight to restrict blood circulation. However, it should be understood that such a tapered shape is preferred but not by way of limitation, and that any other shape can be utilized in the presently disclosed and claimed inventive concept(s) as long as a toe nail 11 can be inserted into a device 10 and tightly sealed. In addition, a device 10 can also be tapered in a manner such that a center portion of the device 10 has a slightly greater width than ends 13 and 14. By providing the center portion with a greater width (not shown), the device 10 may better fit over the knuckle or joint of a toe nail 11.

Before the device 10 is utilized, an antifungal medicine is applied around the infected toe nail 11 and its surrounding skin areas. The application can be performed using any type of applicators, including but not limited to, fingers, brushes, swabs, etc. An effective amount of the antifungal medicine should be used to form a thin layer on the infected nail surface(s). The antifungal medicine can also be applied on the infected areas of the skin surrounding the nail(s). Immediately after application of the antifungal medicine, the infected toe nail 11 is inserted into the interior cavity 12 of the device 10 as shown in FIG. 1. The device 10 may be left in position around the infect toe nail 11 for any amount of time sufficient for treatment thereof, for example but not by way of limitation, for at least about 12 hours. Then the device is taken off and disposed. After removal of the used device, a new device can be positioned on the toe nail 11 as described herein above. The infected toe nail 11 may be inspected every day until new nails are grown without any infections.

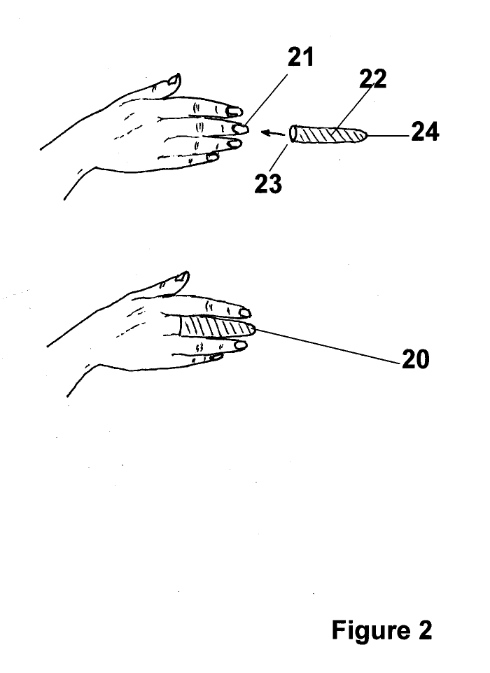

FIG. 2 shows an exemplary embodiment of an oil resistance polymer device 20 before and after its application to an infected finger nail 21 in accordance with the presently disclosed and claimed inventive concept(s). The device 20 comprises an enclosure sized for housing the finger nail 21. The enclosure comprises an integrally molded elastomeric polymer sheath having an interior cavity 22, an open end 23 and a closed end 24. The interior cavity 22 substantially conforms to the size and shape of the finger nail 21 and its surrounding skin areas. The device 20 may have a slightly tapered shape such that the open end 23 is narrower than the closed end 24. Such a tapered shape combined with the interior cavity allows the device 10 to better fit the finger nail 21 well with a substantially tight seal, but not too tight to restrict blood circulation in the finger. However, it should be understood that such a tapered shape is preferred but not by way of limitation, and that any other shape can be utilized in the presently disclosed and claimed inventive concept(s) as long as a finger nail 21 can be inserted into a device 20 and tightly sealed. In addition, the device 20 can also be tapered in a manner such that a center portion of the device 20 has a slightly greater width than ends 23 and 24. By providing the center portion with a greater width (not shown), the device 20 may better fit over the knuckle or joint of a finger nail 21.

Similarly as described above herein, an antifungal medicine is applied around the infected finger nail 21 and it's surrounding skin areas before the device 20 is utilized. The application can be performed using any type of applicators as described previously. An effective amount of the antifungal medicine should be used to form a thin layer on the infected finger nail surface(s). The antifungal medicine can also be applied on the infected area of the skin surrounding the nail(s). Immediately after application of the antifungal medicine, the infected finger nail 21 is inserted into the interior cavity 22 of the device 20 as shown in FIG. 2. The device may be left in position about the infect finger nail 21 for any amount of time sufficient for treatment thereof, for example but not by way of limitation, for at least about 12 hours. Then the device is taken off and disposed. After removal of the used device, a new device can be positioned on the finger nail 21 as described herein above. The infected finger nail 21 may be inspected every day until new nails are grown without any infections.

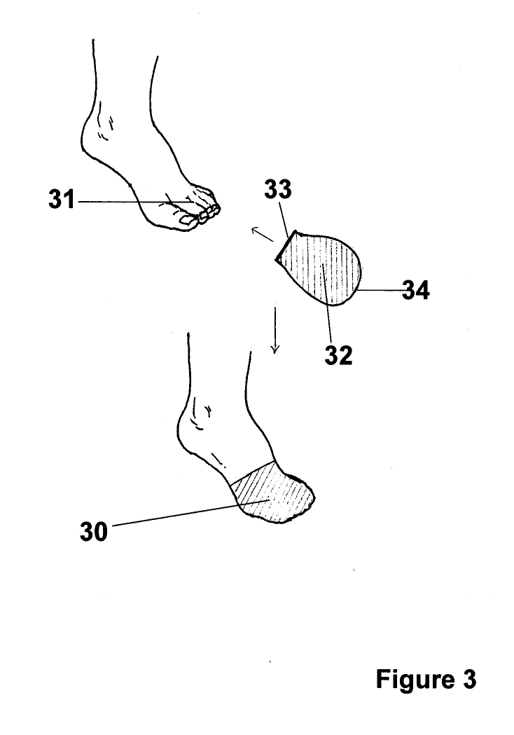

Referring to FIG. 3, another exemplary embodiment of the presently disclosed and claimed inventive concept(s) is described. An elastomeric polymer device 30 is used to treat a fungal infected forefoot (front part of a foot including five toes). The device 30 comprises an enclosure made to fit the whole forefoot 31, and the device 30 has an interior cavity 32, an open end 33 and a closed end 34. The device 30 may have a slightly tapered shape such that the open end 33 is narrower than the closed end 34, which allows the device 30 to better fit the whole infected forefoot with a substantially tight seal, but not so tight as to restrict blood circulation.

The device 30 can be used in a similar manner as the device 10, except that the application of antifungal medicine should be sufficient to form a thin layer on the entire infected forefoot 31. The infected forefoot 31 is inspected every day until new nails and skin without any infection are grown.

The presently disclosed and claimed inventive concept(s) also provides a method of treating at least one infected nail of at least one digit of a subject using the device containing an antifungal treatment composition and a wax described previously. A method comprises: (a) disposing the device over the at least distal end of the at least one digit of the subject having at least one infected nail; and (b) tightly sealing the device around the at least distal end of the at least one digit of a subject having the at least one infected nail.

Methods according to the presently disclosed and claimed inventive concept(s) can be used to treat fungal infected nails and surrounding skin areas. Generally, application of an antifungal medicine and an elastomeric polymer device to a fungal infected nail and its surrounding skin areas can be performed daily or for intermittent intervals, such as for two to three times per week. The duration of treatment can vary greatly, depending upon the degree of severity of an infected nail, the part of the body where the infected nail is treated, the age of the person, the thickness of the nail, the rate of nail growth and the like. Usually, the toe nails of a younger person can be expected to receive treatments up to 6 months, whereas the toe nails of an older person can be expected to receive treatment up to about one year. These periods reflect the time required for infected toe nails to completely grow out of the toe. Treatment of finger nails can be expected to be faster, since finger nail growth is approximately twice as fast as toe nail growth. Effectiveness of the treatment can be evaluated by the subsidence or disappearance of symptoms. Less severe cases where only part of the distal portion of the nail is infected can be expected to require less time for treatment.

Usually, the nail surface will be occluded for a time sufficient to permit full replacement growth of a new nail. Thus, for finger nails, the treatment period will typically last at least 16 weeks, usually lasting at least 24 weeks, and frequently lasting 40 weeks or longer. For toe nails, the treatment period will typically last at least 35 weeks, usually lasting at least 40 weeks, and frequently lasting 60 weeks, or longer.

In one or more embodiments, a therapeutic kit for treating at least one infected nail of at least one digit of a subject is provided. The kit comprises a device comprising an enclosure sized for housing at least a distal end of at least one digit of a subject having at least one infected nail, the enclosure comprising an integrally molded elastomeric polymer sheath having an interior cavity, the interior cavity substantially conforming to the size and shape of the at least distal end of the at least one digit of the subject having the at least one infected nail, and the sheath having an open end and a closed end and tapering from the closed end to the open end; and an antifungal treatment composition. The device and the antifungal treatment composition are described herein above. By combining the antifungal treatment composition and the device, one attains a highly usable, optimal and effective antifungal agent.

The kit in the presently disclosed and claimed inventive concept(s) further comprises an applicator and written instructions for using the kit components in at least one method of the presently disclosed and claimed inventive concept(s). The applicator is used for applying the antifungal treatment composition before utilizing the device in the kit. The applicator can be any type of instruments applying the antifungal treatment composition on at least one infected nail of at least one digit of a subject. Examples of the applicator include, but are not limited to, brushes, swabs, pads, rods, felts, gauzes, etc.

All the components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in the field of treating fungal infections. As used herein, the term “package” refers to a suitable solid matrix or material such as glass, plastic, paper, foil, and the like, capable of holding the individual kit components. Thus, for example, a package can be a glass vial used to contain an antifungal treatment composition. The packaging material generally has an external label which indicates the contents and/or purpose of the kit and/or its components.

An oil resistant elastomeric polymer device of the presently disclosed and claimed inventive concept(s) delivers an active additive for therapeutic purposes. The device, the kit and the treatments of the presently disclosed and claimed inventive concept(s) are characterized by little or no recurrence of infection in untreated nails and minimum or no spreading of the infection from one nail to another. The oil resistant polymer device keeps oil-based anti-fungal medicine around infected areas at high concentrations and prevents the medicine from spreading to bedding material at night, or socks and/or shoes during the day time. The device also allows better penetration of the antifungal medicine into the nail structure to achieve an accelerated curing rate. Moreover, the device helps to isolate the infected areas and prevent them from spreading to their vicinities.

In addition, the device has low oxygen permeability and can substantially inhibit the transfer of oxygen from the outer surface of the device to infected areas. As a result, the sealed device reduces the supply of nutrition (i.e. oxygen) for fungal growth and promotes the development of healthy nails. It has been found that low oxygen can deprive the fungal species responsible for the onychomycosis to grow for a time sufficient to permit full replacement of the treated nails so that all spores and other infective material will totally be eliminated. These synergistic effects surprisingly produce a curing result for onychomycosis.

The following examples illustrate the presently disclosed and claimed inventive concept(s). Each example is provided by way of explanation of the presently disclosed and claimed inventive concept(s), not limitation of the presently disclosed and claimed inventive concept(s). In fact, it will be apparent to those skilled in the art that various modifications and variations can be made in the presently disclosed and claimed inventive concept(s) without departing from the scope or spirit of the invention. For instance, features illustrated or described as part of one embodiment, can be used on another embodiment to yield a still further embodiment. Thus, it is intended that the presently disclosed and claimed inventive concept(s) covers such modifications and variations as come within the scope of the appended claims and their equivalents.

### EXAMPLES

### Comparative Example 1

A female patient in mid fifties had onychomycotic infections on her two toe nails of one foot. The nails were discolored and slightly deformed. A layer of Fungi Nail® medicine solution (Kramer Laboratories, Inc) was applied to the infected nails and surrounding areas every morning for about seven months. The nails showed little improvement, and the treatment was suspended.

### Comparative Example 2

A female patient in her early eighties had onychomycosis disease on two toe nails of both feet for approximately five years. The nail plates were significantly thickened and deformed. Black stripes were developed in one great toe nail plate. Fungi Nail® was applied to the infected areas twice a day for about seven months. The infected nails remained unchanged. The treatment was suspended.

### Example 1

The female patient in Comparative Example 1 applied the Fungi Nail® medicine solution to her infected toe nails, and then two oil resistant polymer devices were carefully inserted over the two toes each morning, and taken off and disposed of at night. She applied the medicine and used the device daily. After 12 weeks, healthy nails with normal appearance were observed. The patient's toe nails had been cured and remained healthy for at least six months.

### Example 2

The female patient in Comparative Example 2 complained that the infected nails caused her great pain while walking. The patient was treated with Fungi Nail® liquid medicine combined with four oil resistant polymer devices. Due to the severity of her condition, the patient's toes were covered with the devices at 24 hrs per day for the first seven days. Each day, she took off the used devices, cleaned the feet, applied the medicine, and covered her toes with new devices. On the eighth day, the oil resistant devices were worn only during the day and removed before bed time. After two weeks of treatment, the patient did not feel pain while walking. In the third week, healthy nails were observed as starting to grow at the bottom of her toe nails.

### Example 3

A sixty-one year old male patient had three toe nails on both feet infected for over three years. The patient's diseased toe nails were treated with Fungi Nail® liquid medicine and then covered with three oil resistant polymer devices. The treatment was repeated every morning, and the devices were removed before bed time. After three weeks' treatment, the color of the infected toe nails became lighter and appeared translucent. Healthy nails at the bottom of the nails were observed.

It is, of course, not possible to describe every conceivable combination of the components or methodologies for purpose of describing the disclosed information, but one of ordinary skill in the art can recognize that many further combinations and permutations of the disclosed information are possible. Accordingly, the disclosed information is intended to embrace all such alternations, modifications and variations that fall within the spirit and scope of the appended claims.

1. A device for treating at least one infected nail of at least one digit of a subject, comprising an enclosure sized for housing at least a distal end of at least one digit of a subject having at least one infected nail, the enclosure comprising an integrally molded elastomeric polymer sheath having an interior cavity, the interior cavity substantially conforming to the size and shape of the at least distal end of the at least one digit of the subject having the at least one infected nail, and the sheath having an open end and a closed end and tapering from the closed end to the open end.

2. The device of claim 1, wherein the enclosure is capable of being tightly sealed around the at least distal end of the at least one digit of the subject having the at least one infected nail.

3. The device of claim 1, wherein the elastomeric polymer sheath comprises an elastomeric polymer film having oil resistance and low oxygen permeability.

4. The device of claim 3, wherein the elastomeric polymer film comprises an elastomeric polymer selected from the group consisting of polybutadiene-co-acrylonitrile, polystyrene terpolymer with butadiene and acrylonitrile, chloroprene rubber, halogenated butyl rubber, epichlorohydrin rubber, fluoroelastomers, perfluoroelastomers, chlorosulfonated polyethylene, polyacrylic esters, copolymers or blends of polyvinyl alcohol, polyvinylidene chloride, polyacrylonitrile, polyamide, polyethylene terephthalate, and combinations thereof.

5. The device of claim 3, wherein the elastomeric polymer film is provided with a thickness of less than about 0.3 mm.

6. The device of claim 1, wherein the enclosure is sized for housing at least the distal ends of two or more digits of a subject.

7. The device of claim 1, wherein the enclosure is sized for housing a forefoot of a subject.

8. The device of claim 1, wherein the interior cavity contains an antifungal treatment composition and a wax.

9. The device of claim 8, wherein the antifungal treatment composition comprises at least one selected from the group consisting of miconazole, ketoconazole, econazole, oxiconazole, clotrimazole, naftifine, terbinafine, amorolfine and undecylenic acid.

10. The device of claim 8, wherein the wax comprises at least one selected from the group of natural wax, synthetic wax, silicone wax, and combinations thereof.

11. A method of treating at least one infected nail of at least one digit of a subject, comprising:
(a) topically administering an effective amount of an antifungal treatment composition to at least a distal end of at least one digit of a subject having at least one infected nail;
(b) disposing a device over the at least distal end of the at least one digit of the subject having the at least one infected nail, the device comprising an enclosure sized for housing the at least distal end of the at least one digit of the subject having the at least one infected nail, the enclosure comprising an integrally molded elastomeric polymer sheath having an interior cavity, the interior cavity substantially conforming to the size and shape of the at least distal end of the at least one digit of the subject having the at least one infected nail, and the sheath having an open end and a closed end and tapering from the closed end to the open end; and
(c) tightly sealing the device around the at least distal end of the at least one digit of the subject having the at least one infected nail.

12. The method of claim 11, wherein the elastomeric polymer sheath comprises an elastomeric polymer film having oil resistance and low oxygen permeability.

13. The method of claim 12, wherein the elastic polymer film comprises an elastomeric polymer selected from the group consisting of polybutadiene-co-acrylonitrile, polystyrene terpolymer with butadiene and acrylonitrile, chloroprene rubber, halogenated butyl rubber, epichlorohydrin rubber, fluoroelastomers, perfluoroelastomers, chlorosulfonated polyethylene, polyacrylic esters, copolymers or blends of polyvinyl alcohol, polyvinylidene chloride, polyacrylonitrile, polyamide, polyethylene terephthalate, and combinations thereof.

14. The method of claim 12, wherein the elastomeric polymer film is provided with a thickness of less than about 0.3 mm.

15. A method of treating at least one infected nail of at least one digit of a subject, comprising:
(a) disposing the device of claim 8 over the at least distal end of the at least one digit of the subject having the at least one infected nail; and
(b) tightly sealing the device around the at least distal end of the at least one digit of the subject having the at least one infected nail.

16. A therapeutic kit for treating at least one infected nail of at least one digit of a subject, comprising:
a device comprising an enclosure sized for housing at least a distal end of at least one digit of a subject having at least one infected nail, the enclosure comprising an integrally molded elastomeric polymer sheath having an interior cavity, the interior cavity substantially conforming to the size and shape of the at least distal end of the at least one digit of the subject having the at least one infected nail, and the sheath having an open end and a closed end and tapering from the closed end to the open end; and
an antifungal treatment composition.

17. The kit of claim 16, wherein the elastomeric polymer sheath comprises an elastomeric polymer film having oil resistance and low oxygen permeability.

18. The kit of claim 17, wherein the elastic polymer film comprises an elastic polymer selected from the group consisting of polybutadiene-co-acrylonitrile, polystyrene terpolymer with butadiene and acrylonitrile, chloroprene rubber, halogenated butyl rubber, epichlorohydrin rubber, fluoroelastomers, perfluoroelastomers, chlorosulfonated polyethylene, polyacrylic esters, copolymers or blends of polyvinyl alcohol, polyvinylidene chloride, polyacrylonitrile, polymide, polyethylene terephthalate, and combinations thereof.

19. The kit of claim 17, wherein the elastomeric polymer film is provided with a thickness of less than about 0.3 mm.

20. The kit of claim 16, wherein the antifungal treatment composition comprises at least one selected from the group consisting of miconazole, ketoconazole, econazole, oxiconazole, clotrimazole, naftifine, terbinafine, amorolfine and undecylenic acid.
